(19) United States
(12) Patent Application Publication
(10) Pub. No.: US 2017/0154418 A1
(43) Pub. Date: Jun. 1, 2017

(54) Method of Labeling Invisible Fluorescence by Visible Light with Self-Correction

(71) Applicant: National Applied Research Laboratories, Taipei City (TW)

(72) Inventors: Chun-Li Chang, Hsinchu City (TW); Wen-Hong Wu, Kaohsiung City (TW); Wen-Tse Hsiao, Hsinchu City (TW); Chi-Hung Huang, Hsinchu City (TW); Rui-Cian Weng, Miaoli County (TW); Te-I Chang, Taipei City (TW); Yih-Sharng Chen, Taipei City (TW)

(21) Appl. No.: 14/953,043

(22) Filed: Nov. 27, 2015

(51) Int. Cl. G06T 7/00; A61B 90/00; A61B 5/00; H04N 9/31; G06T 7/60

(52) U.S. Cl. CPC G06T 7/0012; G06T 7/004; H04N 9/3188; G06T 7/60; G06T 7/0024; A61B 5/0071; A61B 90/361; A61B 90/39; A61B 2090/373; A61B 2090/3937

(57) ABSTRACT

A method is provided to label invisible fluorescence by a visible light. A surgeon gets rid of screen for direct observation without repeated location confirmations between surgical site and onscreen mark. Fluid movement of a fluorescent dye can be observed in a real-time mode. Through projecting a visible-light spot at a fluorescent area at real time, the surgeon observes the fluorescent area the visible-light spot projecting to. Hence, the problem that fluorescence imaging technology must rely on screen to see the location of the fluorescent area is solved. When a patient moves or fluorescent areas changes, an image sensor automatically adjusts an area labeled by a visible-light spot through changing a photographing area with a focus automatically set. In addition, the method adjusts a projecting angle of the visible-light spot with an initial mirror and a final mirror only. Adjustment of lens group is not required the saving money.

### TECHNICAL FIELD OF THE INVENTION

The present invention relates to labeling an invisible fluorescent area; more particularly, relates to instantly projecting a visible-light spot to the invisible fluorescent area for a surgeon to observe the area the visible-light spot projecting to.

### DESCRIPTION OF THE RELATED ARTS

The part of spectra of excited fluorescence of a fluorescent dye (Indocyanine Green, ICG) commonly used in clinical is a near-infrared part, which is invisible and the location of the fluorescent dye can not be observed directly by naked eye. Hence, the problem of the conventional ICG fluorescence imaging technology is that, since the ICG fluorescence is not visible, all images can be seen through screen only and a surgeon must look at the screen to know where the fluorescent area is; yet there is no way to see the surgical field at the same time. Furthermore, since the image is a light-emitting fluorescent image in black and white, the background light is very weak and the surgeon must repeatedly reference the screen to know about the fluorescent area for the surgical site. Hence, this is the biggest inconvenience in the existing fluorescent developing method: the method uses an indirect imaging device. In this regard, it was proposed to use Google glasses of wearable infrared developing device, so that the surgeon can see infrared light (Reference: Liu et al, Surgery, Volume 149, Number 5). It was an attempt to solve the problem of looking up to the screen. However, it did not consider the surgeon's maneuverability and comfortness.

Fluorescent developing devices on the market have also tried to solve the above-mentioned inconvenience. For example, HyperEye Medical Systems (HEMS) have a color camera and an infrared camera installed together in a device. Through image fusion, the surgeon can see the background color and the fluorescent area (Reference: InTech Article by Masaki Yamamoto, et al.) Nevertheless, such mode of operation still could not escape the indirect images.

Another prior art proposed an automatic control method of laser displacement for a laser skin-treatment apparatus. This prior art did not disclose how to deal with the change in distance from the camera with respect to the skin treatment. Another prior art was disclosed, where the device comprised an initial and a final mirror and a few adjusting lens groups are located in between and each of the lens groups includes two lens for angle adjustment, which shows a complex structure.

Traditionally, a surgery is done with eyes, which is the most intuitive operation. But the prior arts do not make the fluorescent area visible.

Hence, the prior arts do not fulfill all users' requests on actual use.

### SUMMARY OF THE INVENTION

The main purpose of the present invention is to provide direct observation for a surgeon as getting rid of the screen without repeated location confirmations between surgical site and onscreen mark and to instantly observe movement of a fluorescent dye fluid, where, through instantly projecting a visible-light spot to a fluorescent area, the surgeon can observe the fluorescent area by observing the position the visible-light spot projecting to.

Another purpose of the present invention is to provide a method of self-correction concerning projection scale for coordination with a zoom lens.

Another purpose of the present invention is to adjust a projecting angle of the visible-light spot through an initial and a final mirror only without adjusting a lens group.

To achieve the above purposes, the present invention is a method of labeling invisible fluorescence by a visible light with self-correction, comprising steps of: (a) setting a correcting plate at an initial position and setting an excited-fluorescence light source to shine on the correcting plate; (b) capturing an image by an optical imaging unit contained in an image sensor; (c) moving the correcting plate to align a center position of the correcting plate to a center position of the image and inputting the image into an image analysis processor to select reference points; (d) positioning a light-spot position controller at a neutral position and projecting a visible-light spot on the correcting plate by a point-like visible light source through an optical device; (e) capturing an image by the optical imaging unit with the image sensor; inputting the image to the image analysis processor to obtain the visible-light spot; through analyzing an error between one of the reference points and the visible-light spot, adjusting initial settings of the light-spot position controller to minimize the error to process accurate alignment of the visible-light spot to the reference points through the center position of the correcting plate; recording a view field of the optical imaging unit and drive-settings of the light-spot position controller; (f) generating a standard round by the light-spot position controller; (g) capturing the standard round by the image sensor to be inputted into the image analysis processor and recording a location of the standard round in the image; and (h) obtaining a spatial mapping relationship between the visible-light spot and the image. Accordingly, a novel method of labeling invisible fluorescence by a visible light with self-correction is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the view showing the preferred embodiment according to the present invention;

FIG. 2 is the view showing the apparatus using the present invention;

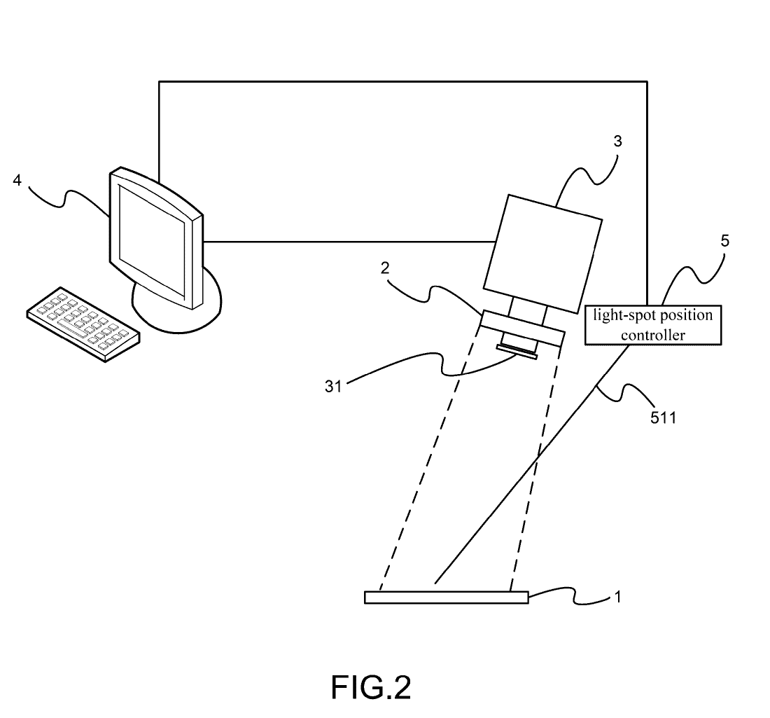

FIG. 3 is the view showing the light-spot position controller;

FIG. 4 is the view showing the projection of the visible-light spot on the correcting plate;

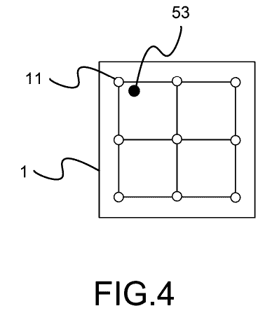

FIG. 5 is the view showing the alignment of the visible-light spot to the reference point on the correcting plate;

FIG. 6 is the view showing the scanning sequence; and

FIG. 7 is the view showing the relationship among the light-spot position controller, the states of the visible-light spot and the image.

### DESCRIPTION OF THE PREFERRED EMBODIMENT

The following description of the preferred embodiment is provided to understand the features and the structures of the present invention.

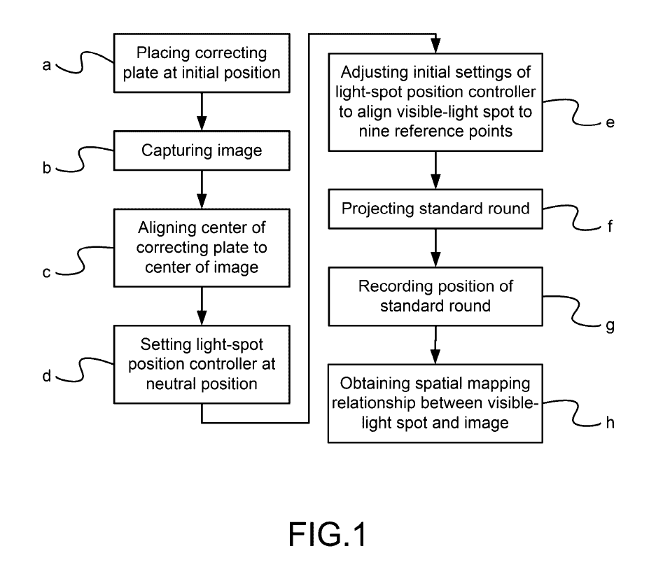

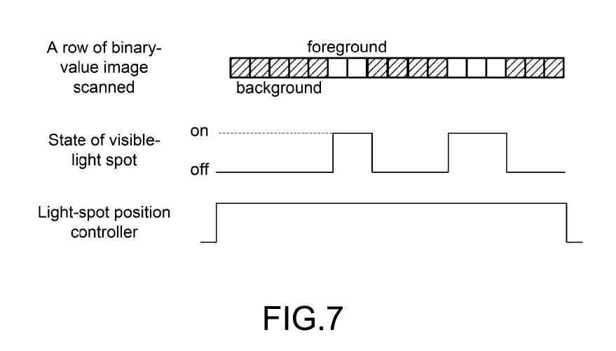

Please refer to FIG. 1˜FIG. 7, which are a view showing a preferred embodiment according to the present invention; a view showing an apparatus using the present invention; a view showing a light-spot position controller; a view showing projection of a visible-light spot on a correcting plate; a view showing alignment of the visible-light spot to a reference point on the correcting plate; a view showing a scanning sequence; and a view showing relationship among the light-spot position controller, states of the visible-light spot and an image. As shown in the figures, the present invention is a method of labeling invisible fluorescence by a visible light with self-correction. The present invention comprises a systematic correction and a dynamic self-correction. The systematic correction displays a spatial mapping relationship for a camera to correct location and size of a projected visible-light spot on image, comprising the following steps:

(a) A correcting plate 1 is placed at an initial position with an excited-fluorescence light source 2 shining on the correcting plate 1.

(b) An image is captured by an optical imaging unit 31 contained in an image sensor 3.

(c) The correcting plate 1 is moved to align its center position to the center position of the image. Then, the image is inputted into an image analysis processor 4 to select reference points 11 from the image for correction.

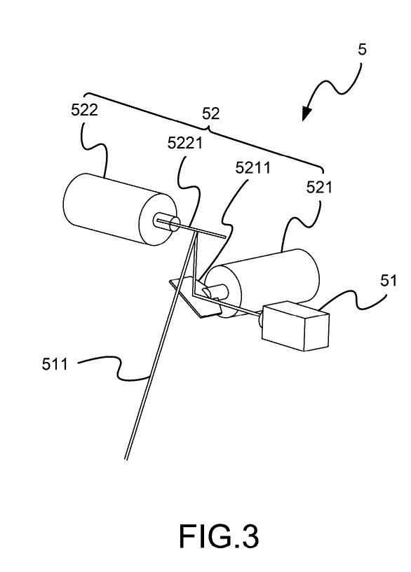

(d) A light-spot position controller 5 is set at a neutral position (x=0, y=0). The light-spot position controller 5 comprises a spot-like visible-light generator 51 and an optical device 52 (as shown in FIG. 3). The optical device 52 comprises a horizontally-scanning device 521 having an initial mirror 5211; and a vertically-scanning device 522 having a final mirror 5221. The spot-like visible-light generator 51 generates a spot-like visible-light source 511 to project a visible-light spot 53 on the correcting plate 1. Therein, a location of the visible-light spot 53 projected on the correcting plate 1 by the spot-like visible-light source 511 is adjusted by moving the initial mirror 5211 of the horizontally-scanning device 521 leftward and rightward and the final mirror 522 of the vertically-scanning device 5221 upward and downward.

(e) The image sensor 3 captures an image by the optical imaging unit 31. The image is inputted into the image analysis processor 4 to obtain the visible-light spot 53. Through analyzing an error between a reference point 11 and the visible-light spot 53, initial settings of the light-spot position controller 5 are adjusted for minimizing the error to process accurate alignment of the visible-light spot 53 to the reference points 11 through the center position of the correcting plate 1. A view field of the optical imaging unit 31 and drive-settings of the light-spot position controller 5 are recorded.

(f) The light-spot position controller 5 projects a standard round 54.

(g) The standard round 54 is captured by the image sensor 3 to be inputted into the image analysis processor 4 for recording a position of the standard round 54 in the image.

(h) A spatial mapping relationship between the visible-light spot 53 and the image is obtained.

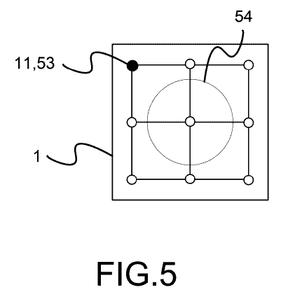

Therein, the correcting plate 1 has nine reference points 11 for correction. Through adjusting the drive settings of the light-spot position controller 5, the visible-light spot 53 is accurately aligned to the reference points 11 (as shown in FIG. 5).

Thus, a novel method of labeling invisible fluorescence by a visible light with self-correction is obtained.

The optical imaging unit 31 is a zoom lens for a dynamical self-correction process, including space correction and self-correction.

For obtaining information of the space correction, a plurality of view distances are uniformly selected between a farthest view field and a nearest view field of the optical imaging unit 31 to separately adjust the light-spot position controller 5 and record a location of the standard round at each view field.

On processing the self-correction, when a user adjusts a magnifying ratio of a lens as changing a view field of the optical imaging unit with the image shrunk, the light-spot position controller 5 is driven to generate the standard round; the location of the standard round in the image is obtained by the image sensor 3; all information of space correction is obtained; the location of the standard round is compared with locations of pre-corrected standard rounds; a position having a smallest distance error to the standard round is obtained; the spatial mapping relationship between the visible-light spot 53 and the image is obtained through interpolation; and the corresponding drive-settings of the light-spot position controller 5 is obtained.

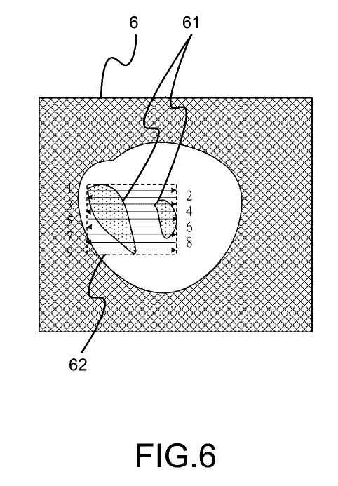

On using the present invention, the actual scanning are shown in FIG. 6 and FIG. 7, where a digital image 6 is captured by the image sensor 3 and fluorescent areas 61 are contained in the image as non-continuous independent blocks.

The fluorescent areas 61 have gray-level values greater than the non-fluorescent background areas. A threshold, which is 255, is set for selecting the fluorescent areas 61 having the gray-level values greater than the threshold. The other areas having the gray-level values smaller than the threshold are labeled as the background areas with their gray-level values set to 0. For avoiding too-small fluorescent areas or noises, a filtering condition is set for selecting fluorescent areas. At last, a smallest rectangle surrounding all fluorescent areas 61 is obtained. As shown in FIG. 6, the light-spot position controller 5 scans the fluorescent areas 61 following a sequence as the numbers shown, which saves time and enhances update frequency.

The threshold mentioned above is determined by a surgeon. Or, all surgeons' experiences are inputted into a database to obtain a recommended value through analysis by the system and the recommended value can be fine-tuned by the surgeon. The filtering condition mentioned above is based on sizes of fluorescent areas, which is determined according to observed-area sizes, organ types, etc. The filtering condition can also be determined according to how many details the surgeon wants to see in the fluorescent areas. Hence, the filtering condition is generally determined by the surgeon.

The present invention allows the surgeon to get rid of the screen for direct observation without repeated location confirmations between surgical site and onscreen mark. The movement of a fluorescent dye fluid can be observed in a real-time mode. Through projecting a visible-light spot to a fluorescent area at real time, the surgeon observes the fluorescent are by observing the visible-light spot projected. Hence, the problem that fluorescence imaging techniques must rely on the screen to see the position of the fluorescent area is solved. Thus, the present invention provides a method for self-correction of projection scale with coordination of a zoom lens. When a patient moves or fluorescent areas changes, an image sensor automatically adjusts an area labeled with a visible-light spot by changing a photographing area with a focus automatically set. In addition, the present invention adjusts an angle of the visible-light spot with an initial and a final mirror only, where adjustment of lens group is not required and cost is thus saved.

To sum up, the present invention is a method of labeling invisible fluorescence by a visible light with self-correction, where a surgeon gets rid of screen for direct observation without repeated location confirmations between surgical site and onscreen mark; the movement of a fluorescent dye fluid can be observed in a real-time mode; through projecting a visible-light spot on a fluorescent area at real time, the surgeon observes the fluorescent area the visible-light spot projecting to; and, hence, the problem that fluorescence imaging techniques must rely on the screen to see the location of the fluorescent area is solved.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of labeling invisible fluorescence by a visible light with self-correction, comprising steps of:
(a) obtaining a correcting plate at an initial position and obtaining an excited-fluorescence light source to shine on said correcting plate;
(b) obtaining an image by an optical imaging unit contained in an image sensor;
(c) moving said correcting plate to align a center position of said correcting plate to a center position of said image and inputting said image into an image analysis processor to select reference points;
(d) positioning a light-spot position controller at a neutral position and projecting a visible-light spot on said correcting plate by a point-like visible light source through an optical device;
(e) obtaining an image by said optical imaging unit with said image sensor; inputting said image to said image analysis processor to obtain said visible-light spot; through analyzing an error between one of said reference points and said visible-light spot, adjusting initial settings of said light-spot position controller to minimize said error to process accurate alignment of said visible-light spot to said reference points through said center position of said correcting plate; recording a view field of said optical imaging unit and drive-settings of said light-spot position controller;
(f) generating a standard round by said light-spot position controller;
(g) obtaining said standard round by said image sensor to be inputted into said image analysis processor and recording a location of said standard round in said image; and
(h) obtaining a spatial mapping relationship between said visible-light spot and said image.

2. The method according to claim 1,
wherein, when said view field of said optical imaging is changed to shrink image size, said light-spot position controller is driven to generate said standard round; said location of said standard round in said image is obtained by said image sensor; all information of space correction is obtained; said location of said standard round is compared with locations of pre-corrected standard rounds; a position having a smallest distance error to said standard round is obtained; said spatial mapping relationship between said visible-light spot and said image is obtained through interpolation; and said corresponding drive-settings of said light-spot position controller is obtained.

3. The method according to claim 2,
wherein, on obtaining said information of space correction, a plurality of view fields between a farthest view field and a nearest view field of said optical imaging unit are uniformly selected to separately adjust said light-spot position controller and record a location of said standard round at each view field.

4. The method according to claim 1,
wherein said light-spot position controller comprises a spot-like visible-light generator and said optical device;
wherein said optical device comprises a horizontally-scanning device having an initial mirror, and a vertically-scanning device having a final mirror;
wherein said spot-like visible-light generator generates a spot-like visible-light source to project said visible-light spot on said correcting plate; and
wherein said spot-like visible-light source projected by said spot-like visible-light generator adjusts a location of said visible-light spot on said correcting plate by moving said initial mirror of said horizontally-scanning device leftward and rightward and said final mirror of said vertically-scanning device upward and downward.

5. The method according to claim 1,
wherein said optical imaging unit is a zoom lens.

6. The method according to claim 1,
wherein said reference points on said correcting plate has nine in number; and
wherein, through adjusting said drive-settings of said light-spot position controller, said accurate alignment of said visible-light spot to said reference points are processed.
